(19) United States
(12) Patent Application Publication
(10) Pub. No.: US 2004/0224922 A1
(43) Pub. Date: Nov. 11, 2004

(54) Use of charged dextran as a mucoactive agent and methods and pharmaceutical compositions relating thereto

(76) Inventor: Malcolm King, Edmonton (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/782,871

(22) Filed: Feb. 23, 2004

Related U.S. Application Data
(63) Continuation-in-part of application No. 09/645,594, filed on Aug. 25, 2000, now abandoned.
(60) Provisional application No. 60/150,605, filed on Aug. 26, 1999.

(51) Int. Cl. A61K 31/715

(52) U.S. Cl. 514/59

(57) ABSTRACT

The present invention is for a charged dextran, preferably dextran sulfate, as an improved mucoactive agent which can be used to improve viscoelasticity and clearance of respiratory tract mucus. The charged dextran can be used in the treatment of animals with impaired mucus clearance, mucus retention and/or mucus hypersecretion, such as cystic fibrosis, chronic bronchitis, bronchiectasis, bronchiolitis and bronchial asthma. Related methods of treatment and pharmaceutical compositions, particularly aerosolized dextran sulfate compositions are encompassed within the scope of the invention.

### RELATED APPLICATIONS

This application is a continuation-in-part of U.S. patent application Ser. No. 09/645,594 filed Aug. 25, 2000 which claims the benefit of priority from U.S. provisional patent application No. 60/150,605, filed Aug. 26, 1999, all of which are herein incorporated by reference

### FIELD OF THE INVENTION

The invention relates to a use of charged dextran, preferably dextran sulfate, as a mucoactive agent and methods and pharmaceutical compositions relating thereto.

### BACKGROUND OF THE INVENTION

Mucus is produced and secreted by animals. The secretion of mucus is a critical component of the defense mechanism of the respiratory tract, trapping inhaled particulate and microbial material for removal via the mucociliary system. When this mechanism fails to clear sufficiently, mucus accumulates, and is coughed up as sputum or retained in the respiratory tract, encouraging colonization by microorganisms, which may lead to chronic lung inflammation and obstruction. Many lung diseases are associated with impaired respiratory mucus clearance, mucus retention and/or mucus hypersecretion, including without limitation, cystic fibrosis, bronchitis, bronchiectasis, bronchiolitis and bronchial asthma Mucus airway obstruction has long been considered the most insidious agent of morbidity and mortality in such diseases, especially in cystic fibrosis.

Airway mucus clearance depends on the physical properties of the respiratory mucous gel as well as interactions between mucus and airflow or mucus and cilia [1]. Mucus retention in the airway usually occurs because of a combination of mucus hypersecretion and impaired mucociliary clearance, and is generally associated with infected mucus and airway inflammation but not always. Impaired mucus clearance may make one more susceptible to infections. Mucoactive medications are treatments designed to enhance the clearance of mucus from the respiratory tract in disorders where mucus clearance impairment is an important feature [2]. Mucokinetic therapy combating mucus retention is a major consideration in the treatment of cystic fibrosis (CF), and other chronic lung diseases in which mucus hypersecretion and impaired airway clearance produce symptoms.

Respiratory tract mucus is a nonhomogeneous, adhesive, viscoelastic gel consisting of water and high molecular weight, crosslinked glycoproteins mixed with serum and cellular proteins (albumin, enzymes, and immunoglobulins), ions and lipids. Mucus of the respiratory tract is quite different from mucus found in other places of the body. It is predominantly carbohydrate (approximately 80% carbohydrate, 20% protein by weight, containing the sugars galactose, N-acetylgalactosamine, N-actylglucosamine, fructose and sialic acid).

The physical, three-dimensional structure that forms the mucous gel is dependent upon a number of forms of bonding: 1) intramolecular disulfide bonds [3, 4]; 2) entanglements with neighboring macromolecules [51]; 3) hydrogen bonds between oligosaccharide side-chains [6]. Although each bond is weak, the number of bond sites make hydrogen-bonding a potentially important target for mucolytic therapy; 4) ionic interactions between fixed negative charges [7, 8]; 5) extra networks of high molecular weight DNA and actin filaments released by dying leukocytes [8-10]. Each of these elements is a potential target for mucoactive therapy.

One of the primary aspects of the current treatment of lung diseases associated with impaired mucus clearance, such as CF, is aimed at changing the physical properties of the mucous gel. For instance, intramolecular disulfide bonds in mucus are the target of classic mucolytic agents such as N-acetylcysteine [3, 4]. Entanglements with neighbouring macromolecules are susceptible to mechanical degradation and can be reduced by high frequency oscillation and other physiotherapy [5].

To date, one of the most successful mucolytic therapies in CF is rhDNase, which has been found to improve lung function in a broad spectrum of patients [13, 14]. The action of DNase is to degrade the three-dimensional network by mucolysis, or molecular weight disruption. Combined with other therapies, there are many possibilities for enhancement of potential benefits of this form of therapy [15].

The other target areas mentioned, such as ionic interactions and hydrogen bonds, have received less attention, but are perhaps no less promising.

Agents that affect ionic charge interactions and hydrogen bonds are not true mucolytics because they alter the crosslink density without reducing polymer chain length, the result of which is a common reduction in both elasticity and viscosity [7, 15].

Ionic agents, such as hypertonic saline, are believed to be mucoactive by shielding the fixed charges along the macromolecular core of the mucin polymer, making it less stiff and less extended and thus reducing the number of entanglement crosslinks with neighbouring macromolecules [8]. However, the tolerability and potential for interfering with bacterial killing may limit the value of hypertonic saline as a therapeutic agent. Nonionic agents such as sugar derivatives reduce the crosslink density of sputum, probably by disrupting the hydrogen bonds between mucin molecules [6]. Among oligosaccharides, neutral dextran, lactose and mannitol have all been considered as potential therapeutic agents [6, 11, 12]. The present inventor with others has shown that low molecular weight neutral dextran (m.w. 4000 or less) is a mucolytic agent, reducing the viscoelasticity and spinnability of CF sputum and improving its mucociliary clearability in in vitro testing and that the effects on viscoelasticity and spinnability were concentration-dependent, being greater at 4% than at 0.4% (wt.tvol.) of dextran [6]. This improved mucocilliary clearability and reduced viscoelasticity was confirmed in in vivo studies [16]. By placing sugar moieties, or oligosaccharides, between mucin-mucin bonds, it is hypothesized that treatment with neutral dextran creates rheologically ineffective mucin-oligomeric sugar bonds, which may lead to the disruption of network crosslinks attributed to hydrogen bonds.

It is hypothesized that agents that might alter more than one type of mucosal bond, such as both the hydrogen bonds and the ionic interactions, may result in improved respiratory tract mucus clearance. The present inventor has found that one such agent, a charged oligosaccharide, low molecular weight heparin, had a greater mucolytic and mucokinetic capacity than the neutral saccharide polymer, dextran. This was seen both in in vitro rheological testing [17] and in excised frog palate clearance measurements [18]. However, heparin is expensive to produce and could potentially have toxic side effects such as pulmonary hemoptysis (bleeding of the tracheobronchial mucosa).

U.S. Pat. No. 5,514,665 teaches that dextrans interfere with adhesion of bacteria (particularly Pseudomonas aeruginosa) to epithelial cells. The patent does not teach anything about effects of dextrans on mucus clearance, mucus viscoelastisity or methods of diagnosing and methods of determining dosage. Attachment of bacteria to epithelial cells is thought to be the first step in infection, particularly in the respiratory tract In conditions such as cystic fibrosis, bacteria such as P. aeruginosa appear to have enhanced capacity to attach and thereby cause disease. This patent is directed to the observation that this enhanced attachment may be attenuated with dextrans. This patent does not describe the use of dextran to alter rheology of mucus or enhance its clearability from the lung. In cystic fibrosis and other chronic respiratory tract conditions, lung injury appears to be enhanced because mucus is abnormally viscous and cannot be readily removed from the airways This is independent from any bacterial-related infections. There is a need for a treatment to improve the rheology of respiratory tract mucus.

Further as the mucoactive agents known to date have limitations, there is a need for an improved mucoactive agent to improve mucus viscoelasticity and clearability.

### SUMMARY OF THE INVENTION

The present invention provides an improved mucoactive agent and methods and uses therefore The improved mucoactive agent is a charged oligosaccharide. Preferably, the charged oligosaccharide is charged dextran Preferably the charged dextran is doxtran sulfate or dextran phosphate. In one embodiment the charged dextran is dextran sulfate. It is herein shown that the charged dextran decreases mucus viscoelasticity and increases mucociliary clearability. The present invention relates to these unexpected findings.

The charged dextran of the invention is used as a mucolytic or mucokinetic agent and acts directly on the rheology of respiratory tract mucus and interferes with the structure thereof. The charged dextran decreases the viscoelasticity and improves respiratory tract mucus clearability from the lung through mucolytic and/or mucokinetic effects. It has this effect even in healthy animals who are free of bacterial infections.

In one embodiment, the present invention relates to a method of decreasing viscoelasticity of mucus from the respiratory tract of an animal by administering to the mucus an effective amount of a charged dextran, preferably dextran sulfate.

In another embodiment, the present invention provides a method of improving mucus clearance in an animal, preferably a human, in need thereof comprising administering to the respiratory tract of the animal an effective amount of a charged dextran, such as dextran sulfate. The animal, in one embodiment a human, in need thereof may have a disease characterized by impaired mucus clearability, mucus retention, and/or mucus hypersecretion, such as cystic fibrosis, chronic bronchitis, bronchitis, bronchiectasis, bronchiolitis, or bronchial asthma. In another embodiment, the disease may be an equine condition such as heaves. However, in one embodiment, the animal in need thereof is not known to have a bacterial infection. In another embodiment, the animal in need thereof does not have a detectable bacterial infection. In another embodiment the animal is free of any bacterial infection.

In one embodiment the charged dextran, preferably dextran sulfate, is administered in the form of a pharmaceutical composition In a preferred embodiment the pharmaceutical composition further comprises a pharmaceutically acceptable carrier, including without limitation diluents and/or excipients. In another embodiment, the pharmaceutical composition is a topical composition and most preferably an aerosol.

Preferably the pharmaceutical composition comprises between about 6.5 mg/ml to 65 mg/ml of a charged dextran, preferably dextran sulfate.

In another preferred embodiment the charged dextran, preferably dextran sulfate is administered in a concentration of between about 6.5 mg/ml to 65 mg/ml charged dextran per composition, preferably per aerosol composition and in one embodiment at a dose of about 6 to 8.5 mls, and preferably about 7 to 8 mls. For instance, when administered by aerosol, in one embodiment the dose is administered over a period of about 15 to 30 minutes. In another embodiment the dose is administered from about 1 to 4 times daily.

In another embodiment, the charged dextran, preferably dextran sulfate, is of low molecular weight, preferably between about 500 (dimer) to about 5000. In another embodiment, the molecular weight of the dextran sulfate is less than 5000.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a bar graph illustrating the effects on tracheal transepithelial potential difference (PD (-mV) by agar bridge technique) in 7 healthy mongrel dogs after 30-minute Ringer aerosol, and after increasing concentrations of aerosolized DexSO₄ (6.5 mg/ml; 20 mg/ml; and 65 mg/ml) in Ringer vehicle. The PD was significantly more negative for all three concentrations of DexSO₄. Values are plotted as the mean±SEM.

FIG. 2 is a bar graph illustrating the effect of Dextran sulfate on Tracheal Mucociliary Velocity (TMV) by charcoal particle displacement, in 7 healthy mongrel dogs during and after 30 minutes of Ringer aerosol, and during and after increasing concentrations of aerosolized DexSO₄ (6.5 mg/ml; 20 mg/ml; and 65 mg/ml) in Ringer vehicle. There were no significant differences in TMV with DexSO₄ administration.

FIG. 3 is a bar graph illustrating the effect of dextran sulfate on Trachcal Mucus Viscoelasticity (TMV) as determined by magnetic rheometry, expressed as the average log G* over 1-100 rad/s, in 7 healthy mongrel dogs during and after 30-minute Ringer aerosol, and during and after increasing concentrations of aerosolized DexSO₄(6.5 mg/ml; 20 mg/ml; and 65 mg/ml)in Ringer vehicle. DexSO₄ administration resulted in a significant decrease in average log G* at 6.5 and 65 mg/ml compared with the respective Ringer control.

FIG. 4 is a linear graph illustrating the effect of dextran sulfate on mucus % solids content (% SC of collected airway secretion in 7 healthy mongrel dogs during and after 30-minute Ringer aerosol and increasing concentrations of DexSO₄ (6.5 mg/ml; 20 mg/ml; and 65 mg/ml). Dextran sulfate induced a significant increase in % SC. Indicates p<0.05 versus Ringer-1; ** Indicates p<0.01 versus Ringer-2.

FIG. 5 is a bar graph illustrating the effect of heparin and dextran sulfate on Tracheal Mucus Viscoelasticity (TMV) of sputum from 7 cystic fibrosis human patients as determined by magnetic rheometry, expressed as the average log G* over 1-100 rad/s after treatment with saline, heparin (MW 6000), Dextran Sulfate (MW 5000, about 17% sulphur by weight), heparin (dimer, highly (fully) sulfated, H-9267: C₁₂H₁₃NO₁₉S₃Na₄), or heparin (dimer, low sulfate, partially de-sulfated H-9142, C₁₂H₁₈NO₁₃SNa) to a final concentration of about 8 mg/ml (or 0.4% wt/vol) of sputum. The highly sulfated heparin dimer showed significant decrease in viscoelasticity as compared to saline control. The effect was similar to heparin, M.W. 6000. The de-sulfated heparin dimer, on the other hand, showed no significant improvement as compared to the control. Dextran sulfate was even more efficient in increasing viscoelasticity than any of the heparins used.

### DETAILED DESCRIPTION OF THE INVENTION

The present invention describes a method of decreasing the viscoelasticity of respiratory tract mucus, comprising contacting the mucus with a mucoactive agent, a charged dextran, such as dextran phosphate or dextran sulfate, more preferably a dextran sulfate, most preferably a low molecular weight charged dextran, such as low molecular weight dextran sulfate.

The administration of the mucoactive agent of the invention to the mucus can be done in vivo or in vitro Possible in vitro applications include, diagnostic purposes, and testing mucus to determine optimum dosage and treatment regimes. For example, a method for diagnosing an animal with impaired respiratory tract mucus clearance could comprise obtaining a sample of the animal's mucus and treating it in vitro with charged dextran, and determining the effect of the charged dextran on the viscoelasticity of the mucus to determine whether the animal may have impaired mucus clearance. Further, a method for determining a dosage regime of an animal with impaired mucus clearance, could comprise:

(a) obtaining a mucus sample from the animal;

(b) subjecting aliquots of the mucus sample to different concentrations of charged dextran;

(c) measuring the viscoelasticity of each of the aliquots of the mucus sample after adminstration of the charged dextran, and

(d) determining the preferred dosage of charged dextran based on comparing the effect of the different concentrations of charged dextran on the viscoelasticity of the mucus sample.

The present invention further describes a method of improving mucus clearance from the respiratory tract of an animal comprising administering to the animal, an effective amount of a charged dextran of the invention, preferably dextran sulfate.

The present invention further describes a method of improving mucus clearance in an animal with impaired mucus clearance, mucus retention and/or mucus hypersecretion, such as animals with lung diseases or conditions including without limitation: cystic fibrosis, chronic bronchitis, bronchitis, bronchiectasis, bronchiolitis (diffuse panbronchiolitis) or bronchial asthma, comprising administering to the animal an effective amount of a charged dextran, preferably dextran sulfate, to an animal in need thereof.

In the studies presented herein low molecular weight dextran sulfate (about m.w. 5000) administered to the respiratory tract in aerosol form through inhalation has been specifically shown to reduce viscoelasticity and increase mucociliary clearability of mucus in the respiratory tract of healthy dogs (dogs with no respiratory tract bacterial infections). It has also been shown to reduce viscoelasticity of mucus obtained from human patients with cystic fibrosis.

The present invention also describes pharmaceutical compositions that can be used in the methods of the invention comprising the mucoactive agent of the invention and a pharmaceutically acceptable carrier and may include, diluents and excipients. “Mucoactive agent” as used herein refers to an agent that can reduce the viscoelasticity of mucus and/or improve or potentially improve the clearance of mucus from the respiratory tract. In one embodiment the mucoactive agent of the invention is a charged dextran. In another embodiment, the mucoactive agent is a low molecular weight charged dextran.

Examples of charged dextrans suitable for use in the present invention include without limitation, dextran phosphate and dextran sulfate Preferably, the charged dextran is dextran sulfate. Charged dextran as used herein may include fully or partially charged dextran. For example, when dextran phosphate or dextran sulfate is used, the degree of phosphorylation or sulfation can vary from low (10-20% of monomer units) to high (fully sulfated or phosphorylated—one sulfate per sugar residue]. Low molecular weight charged dextran, such as low molecular weight dextran sulfate as used herein means charged dextran or dextran sulfate, as the case may be, with a molecular weight of between about 500 (dimer) and 5000. In another embodiment, the low molecular weight dextran is less than 5000.

“Animal” as used herein is any living organism that has a respiratory tract and secretes mucus into the respiratory tract and is susceptible to conditions or diseases involving impaired respiratory tract mucus clearance. Preferably, the animals are mammals. In one embodiment the animals are rats, mice, horses, dogs, or humans In another embodiment, the animal is a dog or a human.

“Effective amount” as used herein means an amount of the mucoactive agent, effective at dosages and for periods of time necessary to achieve the desired result. An effective amount may vary according to factors known in the art, such as the disease state, age, sex, and weight of the animal being treated. Although particular dosage regimes are described in the examples herein, a person skilled in the art would appreciate that the dosage regime may be altered to provide optimum therapeutic response. For example, several divided doses may be administered daily or the dose may be proportionally reduced as indicated by the exigencies of the therapeutic situation.

As preferred administration is by aerosol, dosages by aerosol are generally indicated by concentration of the agent in the nebulizer, and the dose is regulated by the ventilation rate (volume of air per minute) of the animal, which is roughly proportional to body weight. For example, the mucoactive agents of the invention are preferably administered in a concentration of between about 6.5 mg/ml to 65 mg/ml mucoactive agent per composition administered, preferably per aerosol composition and in one embodiment at a dose of about 6 to 8.5 mls (i.e. from about 39 mg to 552.5 mg of mucoactive agent), and preferably about 7 to 8 mls. For instance, when administered by aerosol through inhalation, in one embodiment the dose is preferably administered over a period of about 15 to 30 minutes. In another embodiment the dose is administered from about 1 to 4 times daily.

“Clearance and clearability” as used herein refers to the ability of the mucus to be cleared from the respiratory tract of an animal. This can include without limitation to mucociliary clearance or cough.

“Decreasing viscoelasbcity of respiratory tract mucus” as used herein means a decrease in viscoelasticity as measured by known methods as compared to a starting or initial measurement of viscoelasticity of the respiratory tract mucus of the animal. Similarly with the term “improving”, is any improvement of mucus clearance from a starting level. This would be determined by the ability of a patient to eject mucus from their respiratory tract. The terms would be understood by a person of ordinary skill in the art. It should be noted that even healthy animals and healthy humans can have their mucus clearance accelerated by a decrease in mucus viscoelasticity, further improving mucus clearance or decreasing mucus viscoelasticity even beyond the normal could be of use in compensation for other defects, such as abnormal function of the cilia.

The mucoactive agents of the invention may be administered by topical administration to the mucus or respiratory tract of the animal in a known manner, such as in the form of an aqueous aerosol, dry powder inhaler, metered dose inhaler (with non-aqueous propellant), or direct topical instillation (to intubated patients, or through nasal or sinus irrigation). Most preferably administration is in the form of an aerosol by inhalation.

Depending on the route of administration, the mucoactive agents of the invention can be administered in the form of pharmaceutical compositions which may also comprise pharmaceutically acceptable carriers, diluents and excipients. Examples of suitable pharmaceutical carriers which can be used in the invention are Ringer's solution, water, and sterile saline (0.9% NaCl). The pharmaceutical compositions of the invention can be prepared by per se known methods for the preparation of pharmaceutically acceptable compositions. Suitable methods and pharmaceutical carriers which can be used in the preparation of pharmaceutical compositions of the invention are described in Remington's Pharmaceutical Sciences (Remington's Pharmaceutical Sciences, Mack Publishing Company, Easton, Pa., USA, 1985).

Preferably the pharmaceutical composition comprises between about 6-5 mg/ml to about 65 mg/ml of charged dextran, preferably dextran sulfate.

The present inventor has found that charged dextran, i.e. dextran sulfate, had significant effects on mucus viscoelasticity in in vivo testing, and administration of dextran sulfate by aerosol could increase the rate of mucociliary clearance. Since a charged macromolecule applied to the mucosal surface could alter epithelial ion currents, the changes in tracheal potential difference as an index of epithelial ion transport [191 was also studied. It was shown that dextran sulfate exhibited significant mucolytic capacity. It is believed that the dextran sulfate exerts its effect by modification of both bulk rheological properties and surface effects. As a charged oligosaccharide, dextran sulfate is believed to interfere with hydrogen bond interactions between mucin macromolecules, as well as with ionic interactions in the mucous gel. The net result is a stimulation of mucociliary clearance.

The use of a charged dextran, such as dextran sulfate, as a mucoactive agent has the advantage of being considerably cheaper to produce than heparin or other charged oligosaccharides. Also, dextrans have antimicrobial activities, this with the addition of mucoactive effects has added benefits in the treatment of patients with airway infections. Further, improvement of mucus rheology can minimise injury to the lungs associated with more viscous mucus or impaired respiratory tract mucus clearability. Thus the charged dextran is beneficial to patients without bacterial infections.

Both ionic and nonionic approaches to mucoactive therapy show good promise at selectively improving mucociliary clearance. The use of a charged dextran, such as dextran sulfate, as a mucoactive agent can be combined with other therapies, such as Pulmozyme (rhDNase) and/or N-acetylcysteine derivatives in the treatment of diseases with impaired mucus clearance, such as cystic fibrosis, bronchitis, chronic bronchitis, bronchiectasis, bronchiolitis, and bronchial asthma. For instance, the methods of the present invention using charged dextran, could be combined with a true mucolytic agent (e.g. rhDNase or N-acetylcysteine) that reduces molecular chain length, to improve both mucociliary and cough clearability [8, 15].

The following non-limiting examples are illustrative of the present invention:

### EXAMPLES

### Example 1

The following study was conducted to determine the effect of a charged dextran, dextran sulfate, on the viscoelasticity of mucus and the clearability of mucus from the respiratory tract of healthy dogs.

Materials and Methods

Study Design

Seven healthy mongrel dogs (6 male/1 female), weight 21-28 kg, with no respiratory tract bacterial infections, were studied. The dogs were anesthetized with sodium pentobarbital (ca 30 mg/kg i.v., supplemented as required) and intubated with a cuffed endotracheal tube. They were administered Ringer aerosol first, followed by 6.5 mg/ml dextran sulfate, 20 mg/ml dextran sulfate, and 65 mg/ml dextran sulfate by aerosol. Each aerosolization was of 30 minutes duration, with a rest period of 30 minutes before the beginning of the next aerosol delivery. The aerosols were generated and delivered by a Pari LC STAR nebulizer using an air flow rate of 8 l/min, which results in an output of 0.58 ml/min and particles of 2.0 mm mass median diameter. The nebulizer was loaded in each case with 10 ml of solution. The amount of solution remaining in the nebulizer after 30 minutes was recorded. A T-tube connection was provided to the endotracheal tube during aerosol delivery to ensure a minimal deadspace for ventilation and adequate delivery of the aerosol. The dogs breathed spontaneously throughout the experiment.

Tracheal mucus velocity (TMV) and tracheal potential difference (PD) was measured, and mucus samples were collected for each concentration of delivered drug. TMV was measured twice for each dose, i.e. during aerosolization, between 15 and 25 minutes from the start of aerosol delivery (designated as period-1), and 10-20 minutes after completion of aerosolization (designated as period-2). PD was measured during the period 5 to 10 minutes following each aerosol. Mucus was collected twice for each aerosol, once immediately after 30 minutes of aerosolization and a second time after an additional 30 minutes of aerosolization and a second time after an additional 30 minutes of spontaneous breathing.

All samples of dog mucus collected from endotracheal tubes were placed in 1.5 ml Eppendorf tubes for measurement of viscoelasticity by magnetic rheometry. After initial rheological testing, the mucus samples were preserved at −80° C. for subsequent analysis of collection rate and solids content.

Dextran sulfate, sodium salt (DexSO₄) was obtained from Sigma-Aldrich, St. Louis, Mo. (catalog# D7037, lot#88HO725) for use in this in vivo test. Its nominal molecular weight was 5000, and the sulfur content was 17.7%. DexSO₄ was dissolved in Ringer solution as vehicle. Ringer solution comprises NaCl, as well as KCl and CaCl₂ in approximate physiological concentration as found in plasma.

Transepithelial Potential Difference (PD, -mV); PD was measured by using two flexible microelectrodes (agar bridges of PE 160 size) saturated with KCl connected to calomel half cells, one for reference and the other for testing. These were connected to the high impedance input of a grounded pH meter (Fisher Accumet 925). The reference probe was inserted subcutaneously into the hind leg of the anesthetized dog in a supine position. The measurement probe was guided by bronchoscopy and carefully placed in contact with the epithelium ca 2 cm above the main carina. Care was taken to position the measurement tip to make the slightest contact with the mucosal surface. The PD value was recorded when a steady state, value was achieved and remained stable for a least 30 seconds.

Tracheal Mucociliary Velocity (TMV, mm/min): TMV was determined by observing the rate of charcoal marker particle transport in the lower trachea [20]. Under bronchoscopic visualization, ca, 5 mL of a charcoal suspension (Charac-50 diluted in Ringer's 1:10) was deposited on a lateral surface of the lower trachea 1-2 cm above the carina, and the rate of cephalad progression of the leading edge of the charcoal spot was monitored over the course of 10 minutes by gradually withdrawing the bronchoscope and recording its position relative to the endotracheal tube. TMV was determined twice for each aerosolization period, once during the aerosol, starting after 15 minutes had elapsed, and a second time starting about 10 minutes after the aerosolization had been completed. A T-mnnector with a seal was employed to allow for bronchoscopic visualization during the delivery of the aerosols.

Mucus Collection Weight (MCW, mg/30 min): Tracheal mucus was collected by the endotracheal tube (ETT) method, which involves the removal of mucus adherent to the ETT after extubation [24]. Mucus collections were performed twice after each aerosol, once by immediately removing the ETT following aerosol inhalation, and the second 30 minute after inhalation, after re-installing a clean ETT The mucus samples, which had been frozen, were thawed and weighed by microbalance, to determine MCW, an index of flux, and indirectly of the rate of secretion [20].

Mucus Viscoelasticity Measurements: The magnetic microrheometer technique was used to measure the viscosity and elasticity of the dog mucus samples [21]. A 100 mm steel ball was positioned in a 5-10 mL sample of mucus, and the motion of this sphere under the influence of an oscillating electromagnetic field gradient was used to determine the rheological properties of the mucus. The image of the steel ball was projected via a microscope onto a pair of photocells, whose output was amplified and transmitted to an oscilloscope. By plotting the displacement of the ball against the magnetic driving force, the viscoelastic properties of the mucus were ascertained.

The parameters of mucus viscoelasticity determined were the rigidity index or mechanical impedance, i.e. G*, reported here on a log scale, expressing the average vector sum of “viscosity+elasticity” over the frequency range 1-100 rad/s, and tan d, the mechanical loss tangent, expressing the ratio of viscous to elastic deformation [21]. These viscoelastic parameters can be used to predict mucus clearability by ciliary and cough mechanisms, based on the measured Theological properties and observations of clearance from model studies [22].

Solids content (wet:dry weight ratio): A drying apparatus and microbalance were used to calculate the dry weight and wet weight of the collected secretion, from which the percent solids content (% SC) was calculated. The mucus samples, free of oil, were weighed on previously weighed glass slides. The samples were then dried in a microwave oven (750 W for 30 minutes) and allowed to cool. These dried samples were then reweighed, and % SC was calculated from ratio of dry to wet weight [23].

Statistical analysis: Data from each protocol are presented as mean±standard error (SE) of the mean. To analyze the significance of changes in tracheal mucociliary velocity, potential difference, solids content, and log G* at 1-100 rad/s after each dextran aerosol, the data from each dog were compared with its own control vehicle data. Equality of means was tested by analysis of variance (ANOVA); post hoc analysis of changes from baseline was determined by the two-tailed, paired t-test. The StatView statistical package (Abacus Concepts, Palo Alto, Calif.) was used to carry out these analyses.

Results

All seven dogs completely finished each protocol without significant cough or other side effects during dextran sulfate inhalation, and the bronchial mucosal appearance remained normal.

The osmotic pressures of the DexSO₄ solutions were determined by means of a Wescor model 5500 vapor pressure osmometer The excess osmolarity over Ringer's was as follows: 6.5 mg/ml: 15 mOsm; 20 mg/ml: 45 mOsm; 65 mg/ml: 141 mOsm The delivered volume of aerosol from the Pari LC STAR nebulizer averaged 9.0±0.8 ml, and did not change with increasing dose.

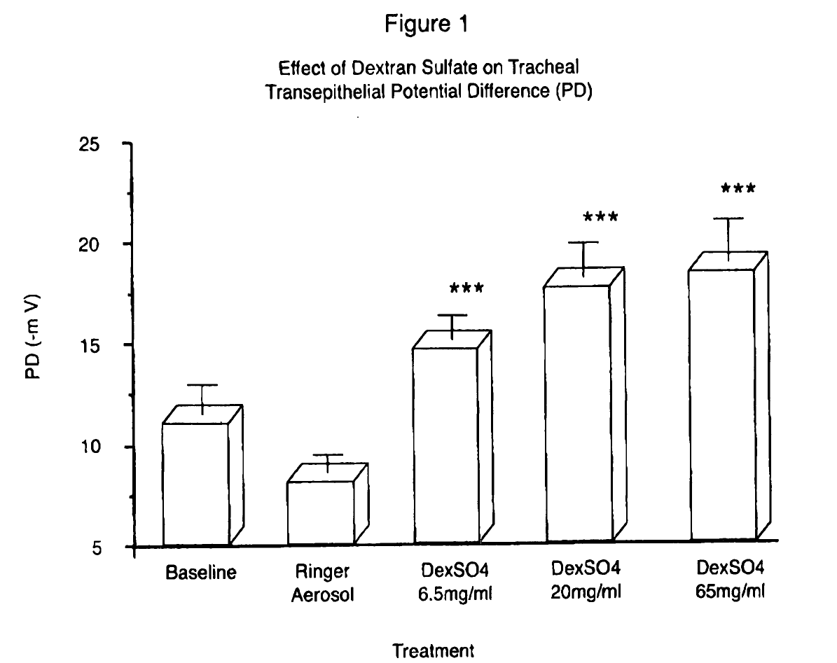

Immediately following 30 minutes of the low-dose DexSO₄ aerosol (6.5 mg/ml) and after administration of the second dose (20 mg/ml) and the highest concentration (65 mg/ml), PD increased significantly compared with Ringer control (6.5 mg/ml: p=0.0005; 20 mg/ml: p=0.0056; 65 mg/ml: p=0.0067). The maximal change in PD was seen for the highest DexSO₄ concentration, a mean increase of 9.86 mV at 65 mg/ml (FIG. 1). The differences between the three dextran concentrations were not significant.

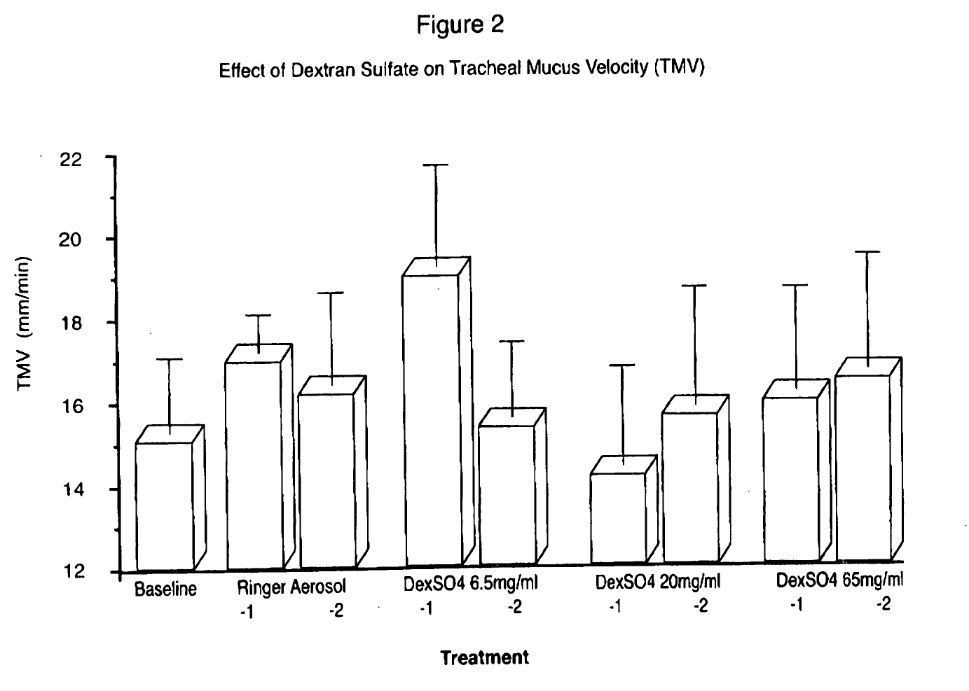

There was no significant change in mucociliary velocity. For the first dose of DexSO₄, at 6.5 mg/ml, a non-significant increase in TMV to 128% of baseline (p=0.066) was observed. Thereafter, TMV remained stable, not differing from baseline with increasing DexSO₄ concentration (FIG. 2).

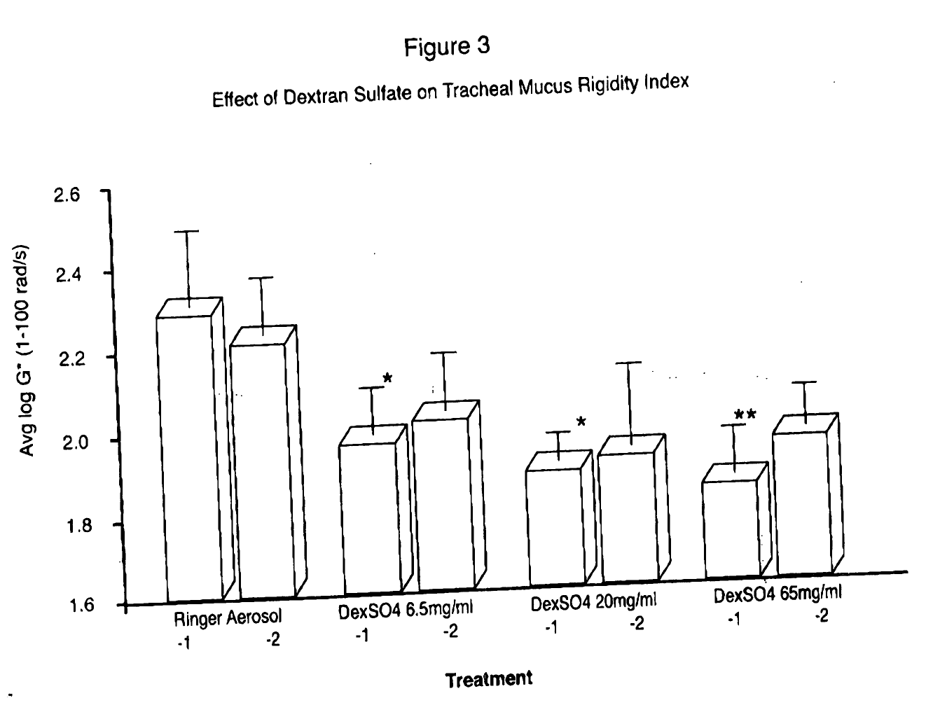

Tracheal mucus viscoelasticity decreased progressively and significantly with increasing concentration of aerosolized DexSO₄. At the highest dose, during 65 mg/ml DexSO₄, the mean decrease in average log G* was 0.42 log units, or a factor of 2.61 (p=0.021) (FIG. 3). The changes in log G* at the three measurement frequencies (1, 10 and 100 rad/s) were all parallel on a logarithmic basis. There were no significant changes in the mucus recoil parameter, tan d, with DexSO₄ administration.

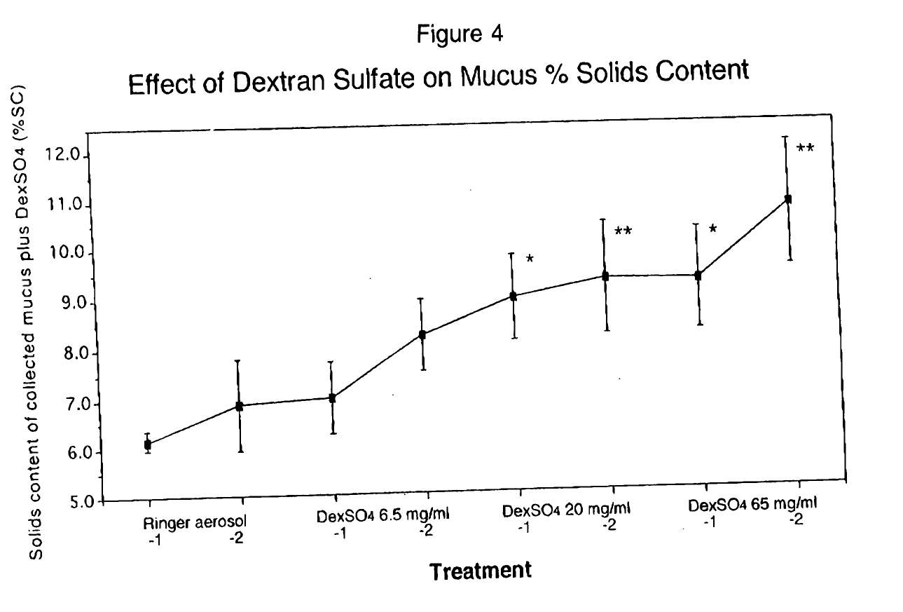

There was no change in mucus collection weight (MCW, mg/30 min) with increasing DexSO₄ concentration, based on the wet weight of collected tracheal fluid (Ringer-1:5.76±0.60 mg; 6.5 mg/ml-1 DexSO₄: 4.35±0.67 mg; 20 mg/ml-1: 4.47±0.59 mg; 65 mg/ml-1: 5.64±1.14 mg). The solids content (% SC) of collected fluid, i.e. for the mucus that accumulated on the ETT during aerosol inhalation, gradually increased during successive 30-minute DexSO₄ aerosols (from 6.16±0.20% at Ringer-1 to 9.27±1.02% at 65 mg/ml-1 DexSO₄, p=0.042; and from 6.87±0.92% at Ringer-2 to 10.78 1.27% at 65 mg/ml-2 DexSO₄, p=0.009) (FIG. 4).

There were significant correlations between mean log G* and PD (p=0.0001), between log G* and % SC (p=0.028), and between PD and % SC (p=0.005).

Discussion

It has been shown that treatment of sputum with sodium chloride increases ciliary transportability on the mucus-depleted bovine trachea [7] and also stimulates lung mucociliary clearance in CF patients, asthmatics, and healthy subjects [24, 25]. It has been shown that agents that alter the ionic interactions or the hydrogen bonding of the mucus gel can produce potentially beneficial effects on mucus clearability This was found to be the case with hypertonic saline [8, 26, 27], and with dextran [6, 16]. Recently, agents that might alter both the hydrogen bonds and the ionic interactions have been studied. It was found that one such agent—the charged oligosaccharide, low molecular weight heparin [17, 18]—had a greater mucolytic and mucokinetic capacity than the neutral saccharide polymer, dextran. The initial conclusion was that heparin decreases mucus viscoelasticity by 1) interaction of its negative charge on the amino groups of the mucin molecule, thereby reducing its entanglement with neighboring sulfate groups, and/or 2) interfering with intermolecular hydrogen bonding due to its short-chain oligosaccharide character (similar to dextran), and/or 3) ionic shielding effects of sodium and heparin on the polylonic moieties of the mucin molecule.

In the present study, it was investigated how low molecular weight dextran sulfate, as a charged oligosaccharide based on dextran, affects tracheal mucus rheology and mucociliary clearance in healthy dogs. Tracheal PD was altered consistently and significantly to higher negative values after DexSO₄ administration. Changes in PD reflect changes in epithelial ion transport or altered integrity [19]. An increase in negative PD value could indicate an increase in epithelial resistance, but in a healthy dog this is unlikely. It more likely indicates an increase in the driving potential for luminal ion transfer, either increased Cl ion secretion or sodium absorption. Given the excess osmolarity of the fluid delivered to the luminal surface, the increase in tracheal PD seen with DexSO₄ suggests an increase in the driving potential for chloride-linked water transfer, indicating that an osmotic mechanism, as suggested by Winters and Yeates [28], was active.

At the same time, there was a progressive and significant decrease in tracheal mucus rigidity with DexSO₄ administration compared with the Ringer vehicle. This decrease in mucus rigidity index is consistent with enhanced mucus clearability by ciliary and cough mechanisms [22]. Although no vehicle control experiment was included in this protocol, previous studies have indicated that repeated administration of Ringer's solution results in no significant change in either TMV or log G* over the course of 4 hours [16]. In the present study, these was a correlation between log G* and PD, with G* decreasing as PD increased. There was also a significant correlation between log G* and % SC, indicating that the viscoelastic effect was proportional to the amount of DexSO₄ delivered and still resident in the airway fluid. In other studies, correlations between the change in PD and the change in mucus viscoelasticity in dogs receiving Nacystelyn aerosol [29] and in mice administered methacholine or uridine triphosphate [30] have been reported. These findings suport a commonality in the relationship between the stimulation of transepithelial chloride transport by these diverse agents and hydration of the mucous gel.

In a previous study [16] the TMV was measured 5-15 minutes after each aerosolization, and thus the peak TMV may have been missed resulting in an underestimation of the maximal drug effect. Accordingly, the protocol of the present study was modified to measure TMV both during and after aerosolization in order to be more certain about this issue. In the present study, using aerosolized DexSO₄, it was found that there was no significant difference in the TMV measured during aerosolization compared with the TMV measured 5-15 minutes after. The fact that no significant increase in TMV was seen in these healthy dogs, despite the improvement in mucus rheological properties, might be ascribed to their healthy status and the probable fact that their TMV was already near optimal. More noticeable improvements in clearance associated with a similar change in mucus rheology might be revealed by studies in a pathological animal model [31].

Based on the wet weight results, DexSO₄ aerosol did not induce an increase of the volume output of tracheal fluid in this dose-dependency experiment, i.e. it did not cause hypersecretion. This was a concern because the protocol involved delivery of a hypertonic aerosol. The solids content of the secretion (% SC) that accumulated on the endotracheal tube cuff during the 30 minutes of aerosolization increased in proportion to the concentration of the delivered dextran sulfate. This increase in nonevaporable solids content is unlikely due to an increased concentration of macromolecules in the mucous gel, since this would lead to an increase in rigidity index, which was opposite to what was observed. In fact the solids content correlated inversely with log G*. The most likely explanation for the excess solids content in the collected secretion is that it contained dextran sulfate which had accumulated in the mucus during aerosolization and did not diffuse away at least for the 60-minute time course between successive DexSO₄ administrations. In a previous study, it was reported that neutral dextran had a significant retention time in the mucus, with a half-life perhaps of the order of a half-hour [16]. This is in sharp contrast to the effects of hypertonic saline aerosol, where less than 30 minutes after delivery of 12% NaCl, no elevation of sodium ion content or excess solids content could he detected [25, 27]. Retention of dextran sulfate in the mucus layer beyond the immediate period of aerosolization, and thus prolongation of the Theological benefit, would be a distinct advantage in terms of therapy.

The present study demonstrates that low molecular weight dextran sulfate applied in vivo significantly reduces the viscoelastic modulus of healthy dog mucus, and increases the predicted mucociliary and cough clearance at a half-log lower concentration range than was observed for neutral dextran [6].

It is also similar to low molecular weight heparin, which from preliminary considerations is effective at a half-log lower concentration than neutral dextran [32].

Although tracheal mucociliary clearance did not significantly increase in these healthy dogs, it did not diminish, and there was no indication of any deleterious effects. The osmotic burden of the delivered aerosol was modest.

Although, no toxic effects of dextran sulfate were observed in the present study. In other laboratories, DexSO₄ has been used to induce experimental colitis in rodents [33]; the mechanism probably involves erosion of the protective intestinal mucus layer. To induce colitis, DexSO₄ (m.w. 40,000) was administered to rats at 5% concentration in drinking water for 7 days. However, dextran sulfate (m.w. 7000) has also been administered orally to human subjects, with little anticoagulant effect because of its poor absorbablity, and no other apparent safety concerns [34]. The potential of dextran sulfate as a therapy with no or minimal toxic effects is a question of dose, route of administration, molecular weight, and charge.

In summary, it can be concluded that delivery of aerosolized dextran sulfate to canine airways leads to reduced viscoelasticity and improved clearability of the tracheal mucus. It thus may be of benefit in patients with CF lung disease or other respiratory diseases where mucus retention is an important feature.

### Example 2

The following study was conducted to compare the affect of dextran sulfate and heparin on the viscoelasticity of mucus and the clearability of mucus from the respiratory tract.

(a) In vitro Sputum from 7 cystic fibrosis human patients were measured with regards to viscoelasticity after treatment with saline, heparin (MW˜6000), dextran sulfate (MW˜5000), heparin (dimer, highly sulfated), or heparin (dimer, de-sulfated) in accordance with the protocols for measuring viscoelasticity described in Example 1. The final concentration of oligosaccharide or disaccharide in the sputum was 8 mg/ml.

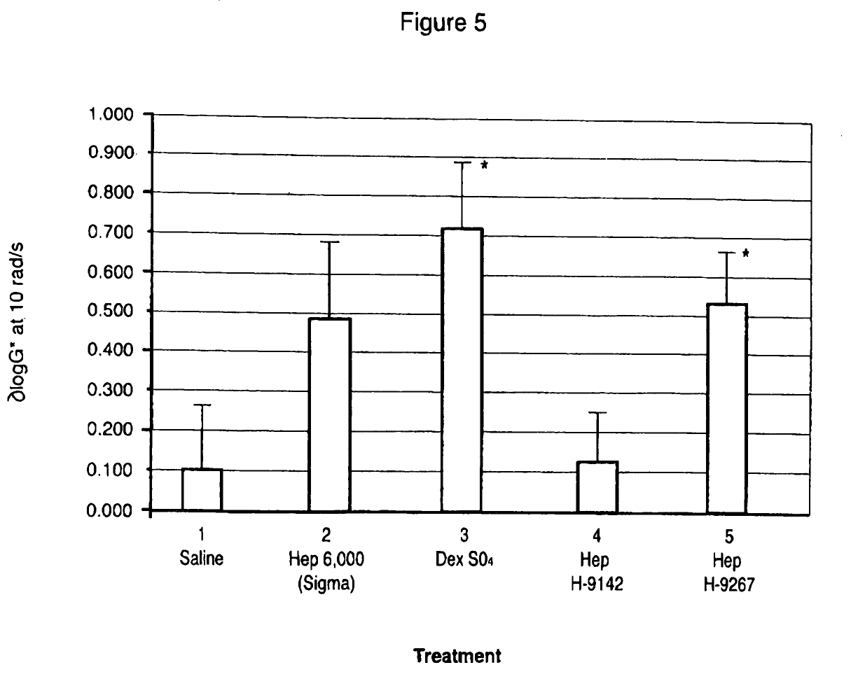

The results of the study are illustrated in FIG. 5 where it can be seen that the highly sulfated heparin dimer showed significant decrease in viscoelasticity as compared to saline control. The effect was similar to heparin, MW 6000. The de-sulfated heparin dimer, on the other hand, showed no significant improvement as compared to the control. Dextran sulfate was even more efficient in increasing viscoelasticity than either heparin, MW 6000 or the highly sulfated heparin dimer

(b) In vivo Seven healthy dogs were anesthetized, intubated and treated via aerosol with heparin sodium (MW 5000-6000) or dextran sulfate (MW 5000) and mucus viscoelasticity, transepithelial potential difference (PD) and tracheal mucus velocity (TMV) were determined, in accordance with the protocols described in Example 1 above. Non-lactated Ringer's solution was used as control. Heparin and dextran sulfate showed similar effects on mucus viscoelasticity and TMV. For heparin at 6.5 mg/ml, there was a significant decrease in PD, to 55.1% of control, and at this same dose, mucus viscoelasticity (G*) was significantly decreased to 36% of control. These changes were accompanied by a nonsignificant, upward trend in TMV, to approximately 130% of control. However, changes might be even more pronounced in animals with initially rigid mucus.

The above studies illustrate that dextran sulfate is potentially more effective than heparin as a mucoactive agent. Further, dextran sulfate is less expensive than heparin. No toxic affects were observed at the concentrations employed in these studies.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### Full Citations for References Referred to in the Specification

1. King, M., and B. K. Rubin. 1996. Mucus physiology and pathophysiology: Therapeutic aspects. Chapter 13 of: Derenne, J. P., W. A. Whitelaw, and T. Similowski, eds. Acute Respiratory Failure in COPD (Lung Biology in Health and Disease Series) Marcel Dekker, New York, 391-411.

2. Rubin, B. K., R. P. Tomkiewicz, and M. King. 1997. Mucoactive agents: Old and new. Chapter 7 of: Wilmott, R. W., ed. The Pediatric Lung. Birkhduser, Basel, 155-179.

3. Sheffner, A. L. 1963. The reduction in vitro in viscosity of mucoprotein solutions by a new mucolytic agent, N-acetylcysteine. Ann. N. Y. Acad. Sci. 106:298-310.

4. Dasgupta, B., and M. King. 1996. Reduction in viscoelasticity of cystic fibrosis sputum in vitro with combined treatment by Nacystelyn and rhDNase. Pediatr. Pulmonol. 22:161-166.

5. App, E. M., R. Kieselmann, D. Reinhardt, H. Lindemann, B. Dasgupta, M. King, and P. Brand. 1998. Sputum rheology changes in cystic fibrosis lung disease following two different types of physiotherapy: Flutter vs. autogenic drainage. Chest 114:171-177.

6. Feng, W., H. Garrett, D. P. Speert, and M. King. 1998. Improved clearability of cystic fibrosis sputum with dextran treatment in vitro. Am. J. Respir. Cit. Care Med. 157:710-714.

7. Wills, P. J., R. L. Hall, W. M. Chan, and P. J. Cole. 1997. Sodium chloride increases the ciliary transportability of cystic fibrosis and bronchiectasis sputum on the mucus-epleted bovine trachea. J. Clin. Invest. 99:9-13.

8. King, M., B. Dasgupta, R. P. Tomkiewicz, and N. E. Brown. 1997. Rheology of cystic fibrosis sputum after in vitro treatment with hypertonic saline alone and in combination with rhDNase. Am. J. Respir. Crit. Care Med. 156:173-177.

9. Shak, S., D. J. Capon, R. Heilmiss, S. A. Marsters, and C. L. Baker. 1990. Recombinant human DNase I reduces the viscosity of cystic fibrosis sputum. Proc. Natl. Acad. Sci. U.S.A. 87:9188-9192.

10. Vasconcellos, C. A., P. G. Allen, M. Wohl, J. M. Drazen, and P. A. Janmey. 1994. Reduction in sputum viscosity of cystic fibrosis sputum in vitro by gelsolin. Science 263:969-971.

11. Daviskas, E., S. D. Anderson, J. D. Brannan, H. K. Chan, S. Eberl, and G. Bautovich. 1997. Inhalation of dry-powder mannitol increases mucociliary clearance. Eur. Respir. J. 10;2449-2454.

12. Shibuya, Y., P. J. Wills, S. Kitamura, and P. J. Cole. 1997. The effects of lactose on mucociliary transportability and rheology of cystic fibrosis and bronchiectasis sputum. Eur. Respir. J. 10:321s.

13. Fuchs, H. J., D. S. Borowitz, D. H. Christiansen, E. M. Morris, M. L. Nash, B. W. Ramsey, B. J. Rosenstein, A. L. Smith, and M. E. Wohl. 1994. Effect of aerosolized recombinant human DNase on exacerbations of respiratory symptoms and on pulmonary function in cystic fibrosis. N. Engl. J. Med. 33:637-648.

14. Ranasinha, C., B. Assoufi, S. Shak, D. Christiansen, H. Fuchs, D. Empey, D. Geddes, and M. Hodson. 1993. Efficacy and safety of short-term administration of aerosolized recombinant human DNase I in adults with stable stage cystic fibrosis. Lancet 342:199-202.

15. King, M., and B. K. Rubin. 1999. Mucus controlling agents: Past and present. In: Rau, J. L., ed. Aerosolized Drugs for the Respiratory Tract. Respir Care Clinics N Amer. in press.

16. Feng, W., S. Nakamura, E. Sudo, M. M. Lee, A. Shao, and M. King. 1999. Effects of dextran on tracheal mucociliary velocity in dogs in vivo. Pulm. Pharmacol. Ther. 12:35-41.

17. Lee, M. M., and M. King. 1998. Effect of low molecular weight heparin on the elasticity of dog mucus. Clin. Invest Med. 21:S 102.

18. Lee M M, H. Garrett, E. Sudo, W. A. Boyd, and M. King. 1998. Mucociliary clearance increase due to low molecular weight heparin. Pediatr. Pulmonol. 386:S 17.

19. App, E. M., J. G. Zayas, and M. King. 1993, Rheology of mucus and epithelial potential difference: Small airways vs. trachea. Eur. Respir, J. 6:67-75.

20. King, M., S. Kelly, and M. Cosio. 1985. Alteration of airway reactivity by mucus. Respiration Physiol. 62:47-59.

21. King, M. 1988. Magnetic microrheometer. In: Braga, P. C., and L. Allegra, eds. Methods in Bronchial Mucology. Raven Press, New York, 73-83.

22. King, M. 1987. Role of mucus viscoelasticity in cough clearance. Biorheology 24: 589-597.

23. Rubin, B. K., 0. Ramirez, J. G. Zayas, B. Finegan, and M. King. 1990. Collection and analysis of respiratory mucus from individuals without lung disease. Am. Rev. Respir. Dis. 141:1040-1043.

24. Daviskas, E., S. D. Anderson, I. Gonda, S. Eberl, S. Meikle, J. P. Seale, and G. Bautovich. 1996. Inhalation of hypertonic saline aerosol enhances mucociliary clearance in asthmatic and healthy subjects. Eur. Respir. J. 9:725-732.

25. Robinson, M., A. Hemming, J. A. Regnis, D. L. Bailey, M. King, W. Feng, G. J. Bautovich, and P. T. P. Bye. 1998. Improved mucociliary clearance following nebulisation with hypertonic saline in adults with cystic fibrosis. In: Baum, G., ed Cilia, Mucus and Mucociliary Interactions. Marcel Dekker, New York, 265-280.

26. Tomkiewicz, R. P., W. A. Boyd, W. Feng, E. M. App, B. K. Rubin, and M. King. 1997. Tracheal clearance and mucus rheology in healthy dogs after aerosolization of 3% and 7% hypertonic saline. Am. J. Respir. Crit. Care Med. 155:A780.

27. Nakamura S, Sudo E, W. Feng, M. M. Lee, W. A. Boyd, and M. King. 1998. Effects of hypertonic saline aerosolization on tracheal mucus clearance and mucus rheology in healthy dogs. Eur. Respir. J. 12(S28):180s.

28. Winters, S. L., and D. B. Yeates. 1997. Role of hydration, sodium, and chloride in regulation of canine mucociliary transport system. J. Appl. Physiol. 83:1360-1369.

29. Tomkiewicz, R. P., E. M. App, G. T. De Sanctis, M. Coffiner, P. Maes, B. K. Rubin, and M. King. 1995. A comparison of a new mucolytic N-acetylcysteine L-lysinate with N-acetylcysteine: Airway epithelial function and mucus changes in dog. Pulm. Pharmacol. 8:259265.

30. Sudo, E., M. M. Lee, W. A. Boyd, and M. King. 1998. Effect of methacholine and uridine-5′ triphosphate on tracheal mucus rheology in mice. Pediatr. Pulmonol. S 17-229.

31. Tai, S., H. Kai, T. Kido, Y. Isohama, K. Takahama, and T. Miyata. 1997. Effect of human neutrophil elastase on tracheal mucociliary transport in anesthetized quails. Jpn. J. Pharmacol. 75:439-442.

32. King, M., A. Ghahary, R. Franklin, M. Hirji, D. Malchenko, W. A. Boyd, H. Garrett, and M. M. Lee. 1999. Studies on aerosolized low mol. wt. heparin as a mucokinetic agent in dogs. Am. J. Respir. Crit. Care Med. 159:A474.

33. Bjork, S., E. Jennische, A. Dahlstrom, and H. Ahlman. 1997. Influence of topical rectal application of drugs on dextran sulfate-induced colitis in rats. Dig. Dis. Sci. 42:824-832.

34. Lorentsen, K. J., C. W. Hendrix, J. M. Collins, D. M. Kornhauser, B. G. Petty, R. W. Klecker, C. Flexner, R. H. Eckel, and P. S. Lietman. 1989. Dextran sulfate is poorly absorbed after oral administration. Ann. Int. Med. 111:561-566.

### DETAILED FIGURE LEGENDS

FIG. 1

The effects on tracheal transepithelial potential difference (PD by agar bridge technique) in 7 dogs after 30-minute Ringer aerosol, and after increasing concentrations of aerosolized DexSO₄ in Ringer vehicle. The PD was significantly more negative for all three concentrations of DexSO₄. Values are plotted as the mean±SEM.

FIG. 2

Tracheal mucociliary velocity, TMV by charcoal particle displacement, in 7 dogs during and after 30 minute Ringer aerosol, and during and after increasing concentrations of aerosolized DexSO₄ in Ringer vehicle. There were no significant differences in TMV with DexSO₄ administration.

FIG. 3

Tracheal mucus viscoelasticity as determined by magnetic rheometry, expressed as the average log G* over 1-100 rad/s, in 7 dogs during and after 30-minute Ringer aerosol, and during and after increasing concentrations of aerosolized DexSO₄ in Ringer vehicle. DexSO₄ administration resulted in a significant decrease in average log G* at 6.5 and 65 mg/ml compared with the respective Ringer control.

FIG. 4

Solids content (% SQ of collected airway secretion in 7 dogs during and after 30-minute Ringer aerosol and increasing concentrations of DexSO₄. Dextran sulfate induced a significant increase in % SC. * Indicates p<0.05 versus Ringer-1; ** Indicates p<0.01 versus Ringer-2.

FIG. 5

Tracheal mucus viscoelasticity of sputum from 7 cystic fibrosis patients as determined by magnetic rheometry, expressed as the average log G* over 1-100 rad/s after treatment with saline, heparin (MW-6000), dextran sulfate (MW-5000), heparin dimer (highly sulfated), or heparin dimer (de-sulfated). The highly sulfated heparin dimer showed significant decrease in viscoelasticity as compared to saline control. The effect was similar to heparin, MW 6000. The de-sulfated heparin dimer, on the other hand, showed no significant improvement as compared to the control. Dextran sulfate was even more efficient in increasing viscoelasticity than heparin, MW 6000.

What is claimed is:

1. A method of decreasing viscoelasticity of respiratory tract mucus in an animal that does not have a detectable bacterial infection, comprising administering to the respiratory tract mucus of an animal need thereof that does not have a known respiratory tract bacterial infection, an effective amount of charged dextran.

2. The method of claim 1 wherein the charged dextran is a low molecular weight dextran.

3. The method of claim 2 wherein the molecular weight of the dextran is from 500 to 5000.

4. The method of claim 1 wherein the charged dextran is dextran phosphate or dextran sulfate.

5. The method of claim 4 wherein the charged dextran is dextran sulfate.

6. The method of claim 1 for improving respiratory tract mucus clearance from the respiratory tract of the said animal in need thereof.

7. The method of claim 6 for treating an animal with impaired mucus clearance.

8. The method of claim 7 wherein the impaired mucus clearance is associated with a lung disease.

9. The method of claim 8 wherein the lung disease is selected from the group consisting of: cystic fibrosis, chronic bronchitis, bronchitis, bronchiectasis, bronchiolitis and bronchial asthma.

10. The method of claim 7 wherein the animal is a horse that has heaves.

11. The method of claim 9 wherein the animal is human.

12. The method of claim 11 wherein the disease is cystic fibrosis.

13. The method of claim 6 wherein the effective amount is between about 39 mg to about 552.5 mg.

14. The method of claim 6 wherein the charged dextran is administered to the respiratory tract of the animal in the form of a pharmaceutical composition comprising a charged dextran and a pharmaceutically acceptable carrier.

15. The method of claim 14 wherein the pharmaceutical composition is an aerosol and is administered through inhalation.

16. The method of claim 15 wherein the charged dextran is low molecular weight charged dextran.

17. The method of claim 16 wherein the molecular weight of the charged dextran is from 500 to 5000.

18. The method of claim 17 wherein the charged dextran is dextran sulfate.

19. The method of claim 1 for diagnosing an animal with impaired mucus clearance comprising obtaining a sample of the animal's mucus and treating it in vitro with charged dextran, determining the effect of the charged dextran on the viscoelasticity of the mucus to determine whether the animal may have impaired mucus clearance.

20. The method of claim 1 for determining a dosage regime of an animal with impaired mucus clearance, comprising:
(a) obtaining a mucus sample from the animal;
(b) subjecting aliquots of the mucus sample to different concentrations of charged dextran;
(c) measuring the viscoelasticity of each of the aliquots of the mucus sample after administration of the charged dextran, and
(d) determining the preferred dosage of charged dextran based on comparing the effect of the different concentrations of charged dextran on the viscoelasticity of the mucus sample.
